(19) United States
(12) Patent Application Publication
(10) Pub. No.: US 2004/0236239 A1
(43) Pub. Date: Nov. 25, 2004

(54) Monitoring device

(76) Inventors: Jim Murray, Belfast (GB); Peter Donnelly, Castlewellan (GB); John Patrick Howard Lee, Belfast (GB)

Correspondence Address: NATH & ASSOCIATES, 1030 15th STREET, NW, 6TH FLOOR, WASHINGTON, DC, 20005, US

(21) Appl. No.: 10/485,416

(22) PCT Filed: Jul. 31, 2002
(86) PCT No.: PCT/GB02/03526

(30) Foreign Application Priority Data
Jul. 31, 2001 (GB) 0118728.5

(51) Int. Cl. A61B 5/0205; A61B 5/08

(52) U.S. Cl. 600/528; 600/529

(57) ABSTRACT

The present invention provides an apparatus for monitoring breath and heart sounds, the apparatus including, sensors for detecting breath and heart sounds, means for recording breath and heart sounds over time and a pattern classifier for comparing recorded breath and heart sounds with previously recorded breath and heart sounds.

The present invention relates to an apparatus for monitoring breath and heart sounds. In particular the apparatus allows continuous cardio-pulmonary monitoring.

Monitoring of breath and heart sounds is used both in diagnosis and as a means of determining the response of a patient to treatment.

Traditionally monitoring of breath and heart sounds has been effected by a stethoscope. However, there are many instances where the full use and capabilities of the traditional stethoscope are restricted. In particular there may be problems using the stethoscope when, access to the patient is restricted, as in intensive care or operating theatre situations. Also the nature of the patient's condition, for example extensive burns, traumatic injury or obesity can restrict access. Also, if the environment surrounding the patient is noisy (e.g. in ambulances, helicopters, military vehicles, ships, disaster sites etc.) it may be difficult to use a stethoscope effectively.

A further disadvantage of the traditional stethoscope is that it relies on the person using the stethoscope having sensitive hearing across the full frequency range. Interpretation of the sounds produced by a traditional stethoscope relies on the auditory performance of the user. As auditory performance often declines with age, older health professionals using a traditional stethoscope can find it more difficult to correctly interpret the heart and breath sounds produced by a patient. Furthermore, there may be important respiratory and cardiac sounds which are outside the normal auditory range and therefore undetectable by “traditional” stethoscopes.

Cardio-pulmonary monitoring of patients with time is important to determine the response of a patient to treatment and in some cases the progress of disease. Further, cardio-respiratory monitoring of patients at risk from acute illness, including infants and children at risk from Sudden Infant Death Syndrome (SIDS), may enable earlier treatment.

Monitoring of cardio-pulmonary function with time using a traditional stethoscope requires that the health professional is able to detect changes in the heart and breath sounds from individual measurements at particular time points such as each hour, day, week or longer. This relies on the ability of the health professional to recall what a previous measurement sounded like. In addition, if different health professionals are monitoring a patient's cardio-pulmonary function over a time period then the different interpretation of the sounds recorded by each health professional via a stethoscope means that subjective differences in the interpretation of a patient's cardio-respiratory sounds must be taken into account.

An object of the present invention is an improved apparatus for monitoring breath and heart sounds.

Accordingly the present invention provides an apparatus comprising a device including at least two sensors capable of being suitably positioned to allow the capture of breath and heart sounds over a period of time, means for recording the breath and heart sounds and means to analyse the breath and heart sounds.

Suitably the sensors are non-invasive. They may be either disposable or non-disposable. Any sensors which can effectively capture the breath and heart sounds are appropriate ranging from simple microphones to piezo-electric devices, ultrasound devices and accelerometers. They must effectively capture the breath and heart sounds when positioned over the appropriate areas of the patient's chest.

Preferably the device of the present invention comprises at least two sensors positioned such that they are located on each side of the patient's chest.

Preferably a plurality of sensors are suitably positioned for capturing breath and heart sounds by locating the sensors in a matrix. The sensors must be of suitable dimensions to be inserted into this matrix.

Preferably this matrix forms a pad which can be used to suitably locate the sensors by adhesive means.

Alternatively this matrix forms a pad which may be worn or wrapped around a patient to suitably locate the sensors.

The matrix containing the sensors may be made of any suitable material. Typically the matrix containing the sensors is formed from foam, nylon or Gore-Tex material.

Preferably the pad comprises a number of layers.

Preferably the sensors are electronically connected to each other.

In one embodiment the signals produced by the plurality of sensors are transferred to a monitor by a single cable.

Alternatively the signals produced by the plurality of sensors are transferred to a monitor by a wireless interface.

Preferably the matix containing the sensors can be used remotely from the recording means and means to analyse the breath and heart sounds.

Preferably the means for recording the heart and breath sounds can convert the breath and heart sounds into an analogue signal

Preferably the means for recording the breath and heart sounds can convert the heart and breath sounds into a digital signal.

Preferably the means to analyse the breath and heart sounds includes means for determining the geometric position in the body from which the breath and heart sounds originate.

Preferably the means to analyse the breath and heart sounds can convert the breath and heart signal to a graphical output that shows the position of particular sounds in relation to the lung and heart.

Preferably the means to analyse the breath and heart sounds includes means for bandpass filtering the signal in the range 10 Hz-2 kHz.

Preferably the means to analyse the breath and heart sounds includes means for sub-band processing the signal.

Preferably sub band processing of the signal uses two sub-bands up to fn and from fn to an upper frequency limit wherein fn is the anticipated upper frequency limit of the normal range of sound from the transducer site.

Preferably the means to analyse the breath and heart sounds is capable of identifying the rate of respiratory inhalation and exhalation phases.

Preferably the means to analyse the breath and heart sounds includes a pattern classifier to enable the signals recorded to be matched to previously determined breath and heart signals.

Preferably the means to analyse the breath and heart sounds uses short term spectral/parametric analysis of respiratory phases in sub bands.

In one embodiment the sub bands are from 10 Hz to fn and fn to 2 kHz.

The means to analyse the breath and heart sounds can comprise a computer program.

The present invention thus provides a computer program, preferably on a data carrier to a computer readable medium having code or instructions for

a) receiving data from at least one sensor means according to the present invention,

b) generating a pattern from the data of step (a),

c) receiving data from predetermined patterns of breath and heart sounds,

d) matching the pattern derived from step (b) with the predetermined patterns of step (c),

e) displaying the match,

Preferably the device including the sensors to detect breath and heart includes a global positioning satellite locator.

Preferably the device including the senses to detect breath and heart sounds includes further sensors for monitoring the physiological state of the patient.

Examples of such sensors include, but are not limited to, temperature sensors, blood oxygen sensors and other blood gas/chemical sensors.

According to a second aspect of the present invention there is provided a method for interpreting breath and heart sounds comprising the steps of,

(i) positioning of the device including the sensors around the area of interest,

(ii) recording the breath and heart sounds over time,

(iii) converting the breath and heart sounds to a signal in the range of 10 Hz-2 kHz,

(iv) bandpass filtering the signal

(v) identifying the rate of respiratory inhalation and exhalation phases

(vi) comparing the recorded signal data with known signal data of breath and heart sounds,

(vii) determining if the signal data of breath and heart sounds recorded matches known signal data of breath and heart sounds.

Preferably the signal is digital.

Preferably step (iv) consists of performing appropriate filtering and amplification of the signal.

Preferably the method includes the step of sub-processing the recorded signal.

More preferably the method includes the step of mapping the signals to the heart and lung.

An embodiment of the present invention will now be described, by way of example only, with reference to the accompanying drawings,

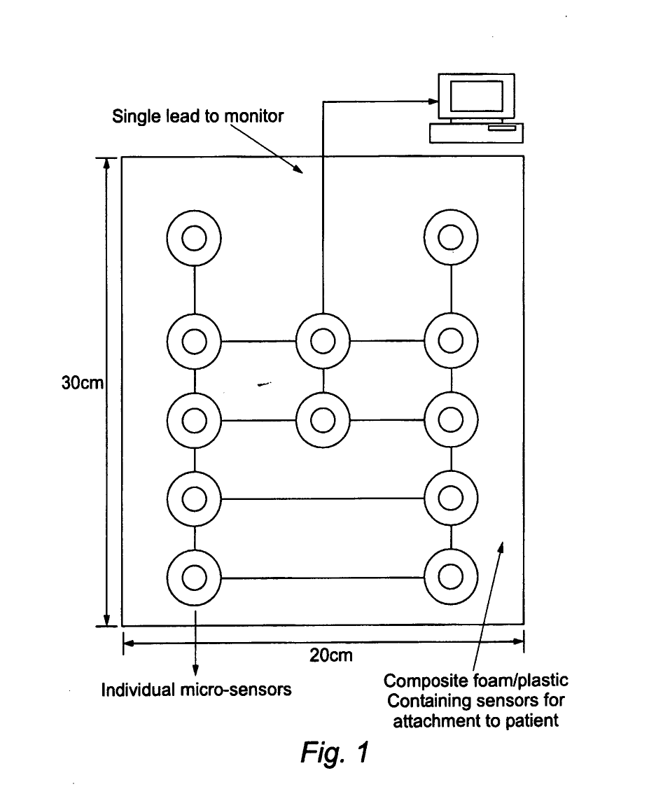

FIG. 1 shows a front view of the device,

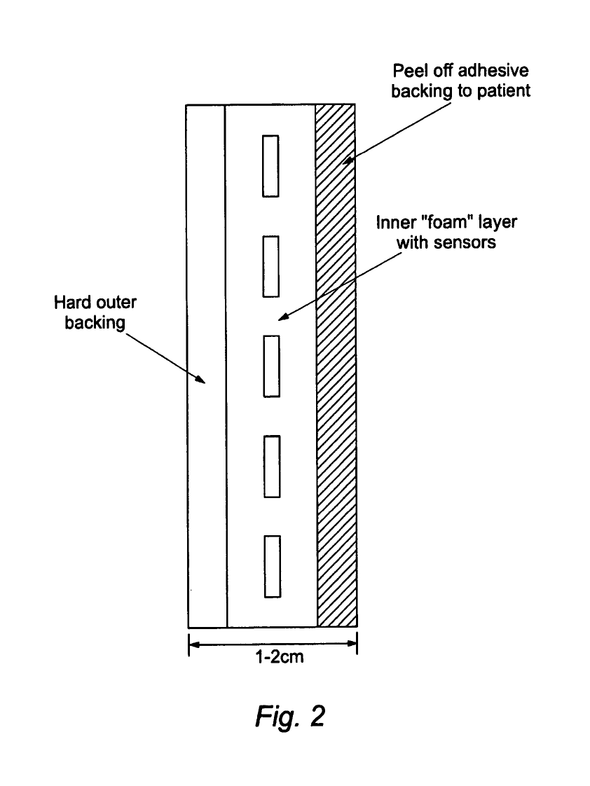

FIG. 2 shows a side view of the device wherein the sensors are mounted in a matrix which is conjoined to an outer layer on one face and an adhesive layer on a second opposite face, and

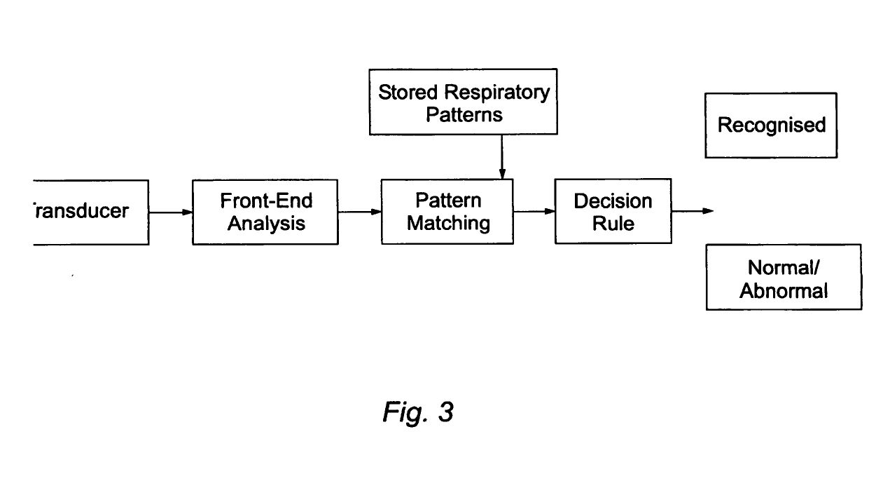

FIG. 3 shows a block diagram of the automatic respiratory recognition system.

With reference to FIG. 1 an embodiment of the present device is a pad comprising a plurality of sensors, typically between six to twelve sensors.

A plurality of sensors may be positioned within each region of the matrix pad with the intention being to capture the strongest “signal” from that region. The use of a plurality of sensors avoids the possibility of single sensor failure preventing measurement of the breath and heart sounds. Thereby accurate information may be relayed to the monitor.

The sensors are arrayed at particular locations in a matrix, the particular locations corresponding to appropriate anatomical positions to enable the continuous capture of breath and heart sounds.

The sensors will effectively “map” the lung and heart. Furthermore, the sensors in the device may capture important respiratory and cardiac sounds which are outside the normal auditory range and therefore undetectable by “traditional” stethoscopes.

The plurality of sensors will provide a complete lung/heart map. As an example if all is well with the patient the sensors will provide an all “green” display and if there are specific diseased areas “red” will be displayed within that region. There will also be varying shades of colour between these two ranges. It is also envisaged that a numerical display will be provided. For example, a range of 0-100, with 0 being the worst and 100 being the best. This may also be expressed as percent.

The pad comprising the matrix in which the sensors are arrayed is typically between 20 cm×30 cm, however the size is dependent on the anatomical proportions of the patient. It can be envisaged that the size of the pad and the location of the sensors may be varied to suit babies or children.

The individual sensors are electronically connected such that the signals produced by each sensor can be transferred to a monitor by a single lead. The monitor enables the amplification, analysis and display of the signals produced by the sensors in both analogue and digital format.

With reference to FIG. 2 the pad is comprised of multiple layers wherein a foam material layer houses the sensors. The foam material layer is attached to a first layer on one face and a second backing layer on the opposite face.

The first layer has an adhesive face, opposite the face of the first layer attached to the foam material layer, for fixing the pad to the patient and locating the sensors to suitable anatomical positions. The adhesive face of this first layer is protected by a peel off protective seal, which remains in place until the pad is to be positioned on to the patient. The adhesive used in the adhesive portion is preferably hypoallergenic, comfortable and sufficiently adherent to allow 2-5 days of continuous placement of the pad.

Between the first layer (in contact with the skin) and the second layer (containing the sensors) it is desirable to have an intermediate “space” or “vacuum” to facilitate and improve sound transmission from the chest to the sensors.

The second backing layer is attached to the foam material layer on the face opposite to that which is attached to the first layer. This second backing layer is thus the furthest from the patient when the pad is positioned on the patient in use. This second backing layer provides strength and robustness to the pad. Further, the second backing layer allows attachment of a lead to the pad for transfer of the signals produced by the sensors to a monitor.

Each sensor in the pad is electronically linked to a common lead for transfer of the signals produced by the sensors to a monitor.

Following the transfer of signals from each of the sensors by the common cable they are amplified, analysed and displayed in both analogue and digital format.

An alternative embodiment of the present invention is also provided wherein the device containing the sensors is not linked to a monitor by a cable, but by a wireless interface system. This wireless interface system allows remote or distant monitoring of the cardio-pulmonary signals.

Using the wireless interface, information can be relayed from the patient to a health professional without the need for the patient to be near a monitor or connected to any equipment other than the sensor containing device.

By suitable positioning of the pad incorporating the wireless interface onto the patient the cardio-pulmonary function of the patient may be monitored. This allows monitoring of patients' cardio-pulmonary function from their own homes, remote locations, or in situations where monitors are not be available, for instance in planes or at sea.

The apparatus can be used to effect the automatic recognition of respiratory sounds.

Respiratory sounds (normal and abnormal) have a typical frequency range of 100-2000 Hz and a dynamic range of some 50-60 dB. The upper extent of the frequency range is dependent upon the point at which the sound is transduced. The sound is effectively low-pass filtered by the body tissue between the lungs and the transducer, with the cut-off frequency of the low-pass fiiltering being dependent of the transducer site.

For digital processing, respiratroy signals should be sampled with a minimum sampling frequency of 4 kHz at a minimum of 8 bits/sample. However, in system and algorithm development stages, a sampling frequeny of at least 8 khz at 16 bits/sample is recommended.

As shown in the block diagram of FIG. 3 of the automatic respiratory recognition system the objective is to automatically determine whether the input acoustic pattern is normal/abnormal and, if abnormal which pathological condition is determined.

The front end analysis involved in the canonic automatic respiratory recognition system is

(1) Bandpass filtering the signal in the range 10 Hz-2 kHz,

(2) Sub-band processing of the signal using two sub-bands—10 to fn and fn to 2 kHz, where fn is the anticipated upper frequency limit of the normal range of sound from the transducer site,

(3) Identification and rate of respiratory inhalation and exhalation phases,

(4) Short-term spectral/parametric analysis of respiratory phases in both sub-bands,

The pattern classifier comprises pattern matching against stored respiratory patterns (based on possible spectral, energy or parametric information) and a decision rule, which may be linear or nonlinear. The pattern classifier can be either a standard statistical classifier or a classifier based on artificial intelligence techniques, such as neural networks or fuzzy logic classifiers.

In use the recorded sounds are transmitted to the analysis means are band pass filtered and sub-band pass filtered.

The recorded sounds also include sounds which are detected and then analysed in real time.

The filtered data is then compared against previously determined data using the pattern classifier.

The previously determined data can be from the same or different patient and may comprise a description indicating if the predetermined data is indicative of normal of abnormal breath and heart sounds.

The newly recorded data can thus be compared against the predetermined data and assigned as normal or abnormal. Further comparison of the recorded data signal with abnormal data might allow a match against a similar previously determined pattern, and such a match may allow a diagnosis of the abnormality and possibly the disease promoting the abnormality to be made by the analysis means.

A global positioning satellite locator (GPS) or further sensors enabling monitoring of the patient may also be incorporated into the pad of the device and the information from the GPS locator or alternative sensor relayed to the monitor by the wireless interface means.

It can be envisaged that the device may be suitably positioned to the patient by alternative means than adhesive.

The sensors may be incorporated into a pad which can be wrapped around the patient or worn by the patient to allow positioning of the sensors at suitable anatomical positions.

Alternatively the sensors may be incorporated with alternative fixing means such as suction cups to allow their accurate placement onto the patient.

The present invention has a number of advantages. It may be used to continuously monitor a patient's cardiopulmonary function. This is advantageous over traditional stethoscopes, which can only record a patient's cardiopulmonary function at distinct time points.

As the device allows the non-subjective monitoring of a patient's cardio-pulmonary function over time, differences in the interpretation of cardio-pulmonary sounds by different health professionals do not have to be taken into account when monitoring the patient.

The device is primarily designed for monitoring breath and heart sounds over the patient's chest, however it could easily be adapted for foetal monitoring either throughout pregnancy or during labour. Similarly, if a woman requires anaethesia/surgery/intensive care during her pregnancy it is not inconceivable that one device could be used to monitor the mother and another to monitor the foetus.

In use the device, which includes in the sensors is a pad which can be wrapped around the patient, the pad is then suitably positioned around the patient chest such that breath and heart sounds can be measured. Due to the plurality of the sensors the exact positioning of the pad is not crucial as typically if placed in a generally correct position, breath and heart sounds will be detected and recorded.

The pad is kept in position for a period of time suitable to allow data collection, this may be minutes, hours or days as required to allow breath and heart sounds to be suitably recorded.

The breath and heart sounds are transmitted to recording means to record the sounds.

Transmission may occur via wires linking the device to the recording and analysis means of via a wireless system.

Further usage and development of the device could be in the field of veterinary obstetrics and veterinary medicine with regard to both large and small animals that are pregnant/about foal, calf etc. or need anaesthesia and surgery.

The device will further provide clear and effective training for students of medicine and nursing, as it will allow the unambiguous interpretation of normal and pathological heart and breath sounds.

The device is robust, easily stored and not easily damaged. The entire device or any part thereof may also be disposable.

It maybe used in daylight or in the dark which is useful in military situations or for use in dark rooms.

As there is an equal distribution of sensors between the left and right sides of the chest, differential interpretation of normal and abnormal breath sounds will be possible.

The device will allow diagnosis or determination of a patient's response to treatment to be performed by a suitable health professional from a distance.

This distant or remote monitoring of a patient's cardio-pulmonary function has particular importance in cases where patients are in planes, ambulances, helicopters or remote situations.

1. An apparatus comprising a device including at least two sound sensors embedded at different locations in a pad capable of attachment to the skin of a patient, the sensors being suitably positioned in the pad to allow the capture of breath and heart sounds over a period of time, means for recording the heart and breath sounds, and means to analyze the heart and breath sounds.

2. An apparatus as claimed in claim 1 wherein the device comprises at least two sensors positioned such that they are located on each side of the patient's chest.

3. An apparatus as claimed in claim 1 wherein a plurality of sensors are suitability positioned for capturing breath and heart sounds by locating the sensors in a matrix.

4. An apparatus as claimed in claim 1, 2 or 3 wherein the pad is attachable to the patient's skin by adhesive means.

5. An apparatus as claimed in any preceding claim, wherein the pad may be worn or wrapped around a patient to suitability locate the sensors.

6. An apparatus as claimed in any preceding claim wherein the sensors are electronically connected to each other.

7. An apparatus as claimed in any preceding claim wherein the signals produced by the plurality of sensors are transferred to a monitor by a single cable.

8. An apparatus as claimed in any preceding claim wherein the sensors can be used remotely from the recording means and means to analyze the breath and heart sounds.

9. An apparatus as claimed in any preceding claim wherein the means for recording the breath and heart sounds can convert the breath and heart sounds into an analogue signal.

10. An apparatus as claimed in claims 1 to 8 wherein the means for recording the breath and heart sounds can convert the breath and heart sounds into a digital signal.

11. An apparatus as claimed in any preceding claim wherein the means to analyze the breath and heart sounds includes means for determining the geometric position in the body from which the breath and heart sounds originate.

12. An apparatus as claimed in any preceding claim wherein the means to analyze the breath and heart sounds can convert the breath and heart signal to a graphical output that shows the position of particular sounds in relation to the lung and heart.

13. An apparatus as claimed in any preceding claim wherein the means to analyze the breath and heart sounds includes means for bandpass filtering the signal in the range 10 Hz to an upper frequency limit.

14. An apparatus as claimed in any preceding claim wherein the means to analyze the breath and heart sounds includes means for sub-band processing the signal.

15. An apparatus as claimed in claim wherein sub-band processing of the signal uses two sub-bands 10 Hz to fn and from fn to an upper frequency limit wherein fn is the anticipated upper frequency limit of the normal range of 17 sound from the transducer site.

16. An apparatus as claimed in any preceding claim wherein the means to analyze the breath and heart sounds is capable of identifying the rate of respiratory inhalation and exhalation phases.

17. An apparatus as claimed in any preceding claim wherein the means to analyze the breath and heart sounds includes a pattern classifier to enable the signals recorded to be matched to previously determined breath and heart signals.

18. An apparatus as claimed in any preceding claim wherein the means to analyze the breath and heart sounds uses short term spectral/parametric analysis.

19. An apparatus as claimed in any preceding claim including a global positioning satellite locator.

20. An apparatus as claimed in any preceding claim including additional sensors for monitoring the physiological state of the patient.

21. A method for interpreting breath and heart sounds using the apparatus of claim 1, comprising the steps of:
i) positioning the device including the sensors around the area of interest,
ii) recording the breath and heart sounds over time,
iii) converting the breath and heart sounds to a signal in the range of 10 Hz to an upper frequency limit,
iv) bandpass filtering the signal
v) identifying the rate of respiratory inhalation and exhalation phases
vi) comparing the recorded signal data with known signal data of breath and heart sounds,
vii) determining if the signal data of breath and heart sounds recorded matches known signal data of breath and heart sounds,

22. A method as claimed in claim 21 including the step of sub-processing the recorded signal.

23. A method as claimed in claim 21 or 22 including the step of mapping the signals to the heart and lung.
